(19) United States
(12) Patent Application Publication
(10) Pub. No.: US 2017/0119250 A1
(43) Pub. Date: May 4, 2017

(54) PORTABLE HARDWARE FIXTURE FOR FUNDOSCOPY

(71) Applicant: THE CHARLES STARK DRAPER LABORATORY, INC., Cambridge, MA (US)

(72) Inventors: Vijaya B. Kolachalama, Cambridge, MA (US); Cort N. Johnson, Cambridge, MA (US); Francis J. Rogomentich, Cambridge, MA (US); Mitchell L. Hansberry, Cambridge, MA (US); Scott T. Bambrick, Cambridge, MA (US); Philip D. Parks, II, Cambridge, MA (US)

(21) Appl. No.: 15/344,231

(22) Filed: Nov. 4, 2016

Related U.S. Application Data
(60) Provisional application No. 62/250,613, filed on Nov. 4, 2015.

(51) Int. Cl. A61B 3/12; A61B 3/00; G02B 7/02; F21V 8/00; A61B 3/14; A61B 5/00

(52) U.S. Cl. CPC A61B 3/1208; A61B 3/14; A61B 5/6898; G02B 7/021; G02B 6/0001; A61B 3/0025; A61B 5/0022; G02B 1/11

(57) ABSTRACT

A fundoscopy system comprises a fixture supporting optics configured to be mounted to a surface of a smartphone and align the optics with a pupil of a camera of the smartphone and to align a light guide with an illumination source of the smartphone. The light guide is configured to direct light from the illumination source into an eye of a subject disposed in front of the fixture. The fixture is further configured to direct light reflected from a retina of the eye through the optics and into the pupil of the camera of the smartphone.

### CROSS-REFERENCE TO RELATED APPLICATION

This application claims priority under 35 U.S.C. §119(e) to U.S. Provisional Application Ser. No. 62/250,613 titled “PORTABLE HARDWARE FIXTURE FOR FUNDOSCOPY,” filed Nov. 4, 2015, which is incorporated herein by reference in its entirety for all purposes.

### FIELD OF INVENTION

Aspects and embodiments disclosed herein relate generally to systems and methods for examining the eyes of a subject for diabetic retinopathy.

### BACKGROUND

According to the 2014 National Diabetes Statistics Report, 29.1 million people in the United States have diabetes, 8.1 million of which are undiagnosed. As recently as 2008, 4.2 million people (28.5% of diabetic adults 40 years of age or older) had diabetic retinopathy (DR) and about 655,000 (4.4%) had advanced DR with conditions such as clinically significant macular edema (ME) and proliferative DR, which are major causes of severe vision loss. It has been shown that early detection and treatment of DR can decrease the risk of severe vision loss in about 90% of the subjects. However, approximately 50% of diabetic subjects do not undergo any form of documented screening exams, leading to an enormous burden on the US healthcare system when symptomatic subjects require expensive late-stage intervention.

### SUMMARY

In accordance with one or more aspects, there is provided a fundoscopy system comprising a fixture supporting optics configured to be mounted to a surface of a smartphone and align the optics with a pupil of a camera of the smartphone and to align a light guide with an illumination source of the smartphone, the light guide configured to direct light from the illumination source into an eye of a subject, the fixture further configured to direct light reflected from a retina of the eye through the optics and into the pupil of the camera of the smartphone.

In some embodiments, the fixture includes a mount having a flat base configured to be disposed against a rear surface of the smartphone.

In some embodiments, the fixture further comprises a plurality of legs that fit around a body of the smartphone and hold the base in place against the rear surface of the smartphone.

In some embodiments, the plurality of legs are formed from a resilient material that deforms when the fixture is mounted on the smartphone and exerts pressure on front and rear surfaces of the smartphone to hold the fixture in place on the smartphone.

In some embodiments, the optics include a beam splitter, objective lens, and eyepiece lens.

In some embodiments, the optics are disposed in a barrel and the fixture further includes a barrel mount configured to retain the barrel.

In some embodiments, one or more of the beam splitter, objective lens, and eyepiece lens has an anti-reflective coating.

In some embodiments, the system further comprises an eyepiece disposed on a front of the fixture.

In some embodiments, the light guide is defined in a body of the fixture and has an axis parallel to a path of light through the optics.

In some embodiments, the light guide terminates at a reflector disposed at an angle of approximately 45 degrees relative to the axis of the light guide.

In some embodiments, the reflector is a mirror.

In some embodiments, the reflector is a reflective inner surface of the fixture.

In some embodiments, the fixture further includes a second light guide extending from the reflector to a beam splitter included in the optics.

In some embodiments, the second light guide is substantially perpendicular to the light guide.

In some embodiments, the fixture further includes one or more light guides configured to direct light from the illumination source of the smartphone to one or more positions at a periphery of an eyepiece of the fixture.

In some embodiments, the one or more light guides comprise fiber optic cables.

In accordance with one or more aspects, there is provided a method of imaging a retina of a subject. The method comprises mounting a fixture supporting optics and a light guide to a surface of a smartphone, aligning the optics with a pupil of a camera of the smartphone, aligning the light guide with an illumination source of the smartphone, the light guide directing light from the illumination source into an eye of the subject disposed in front of the fixture, and receiving light reflected from the retina of the eye through the optics and into the pupil of the camera of the smartphone.

In some embodiments, the method further comprises capturing an image of the retina with the camera of the smartphone.

In some embodiments, the method further comprises analyzing the image for signs of diabetic retinopathy.

In some embodiments, the method further comprises electronically transmitting the image to a remote location for analysis.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing.

In the drawings:

FIG. 1 is a schematic drawing of an embodiment of an optics layout of a smartphone-based fundoscopy system;

FIG. 2 illustrates an exemplary fixture for a fundoscopy system mounted on a smartphone;

FIG. 3 illustrates a partial cross-sectional view of the fixture shown in FIG. 2;

FIG. 4 is an exploded view of the fixture shown in FIG. 2;

FIG. 5 illustrates another embodiment of an exemplary fixture for a fundoscopy system mounted on a smartphone;

FIG. 6 illustrates an experimental system for testing a fundoscopy system as disclosed herein; and

FIG. 7 illustrates an image obtained from a test of a fundoscopy system as disclosed herein.

### DETAILED DESCRIPTION

Applicants have determined that there is a clear need for developing early, efficient, and cost-effective tools for the screening of retinal disease, particularly at the level of primary care or other preventive care settings. Alternate modalities that are more amenable to primary care (or even home use) screening, for example, smartphone-based retinal imaging may help increase the rate of diabetic retinopathy (DR) screening. These tools may remove a fundamental barrier to increased screening, i.e., the clinical visit. Recognizing the need to develop such technologies, Applicants designed a portable hardware fixture that could facilitate smartphone-based fundoscopy. This portable fixture slips easily over the built-in camera on a smartphone, for example, an Apple iPhone®, a Samsung Galaxy S7, or a Google Pixel smartphone, supplementing the existing optics and lighting with additional magnifying optics to capture an image of the retina of a subject. As the term is used herein, a “smartphone” also encompasses mobile computing devices, for example, an Apple iPad® or other tablet computer.

The retinal imager consists of two separate systems for providing illumination and imaging, respectively that interface customized optical components with the smartphone camera. The illumination system uses the light emitting diode (LED) light source from the smartphone to flood the retinal surface useable field-of-view with light. The imaging system relays light reflected from the retina and output from the pupil of the eye to the input pupil of the smartphone camera to produce efficient coupling of the retinal backscattered light signal. The illumination system incorporates a beam-splitter placed in front of the subject's pupil. The imaging system conditions an image signal including a retinal backscattered signal that has been collimated by the eye lens to into a known state. The objective lens is positioned to relay the reflected light output from the eye pupil to the camera pupil with a 1:1 magnification while simultaneously satisfying the required effective focal length for the eye lens-objective lens-eyepiece lens-camera lens combination. Light from the retina is collimated at the input to the cell phone camera and is therefore well suited for imaging with a cell phone camera. In some embodiments, the field of view at the retina covers a 4 mm×4 mm area.

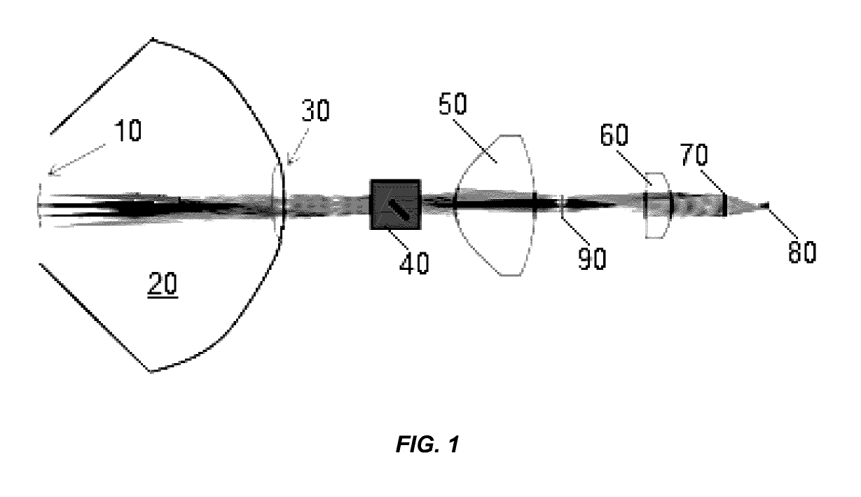

A schematic of one embodiment of the optics layout of a smartphone-based fundoscopy system is illustrated in FIG. 1. The system is designed to image the retinal plane 10 of the eye 20 of a subject through the pupil 30 of the eye 20. Illumination is provided by light emitted from the LED of the smartphone camera (not shown), which passes through a beam splitter 40 and then through the pupil 30 after which it impinges on the retina 10 and is reflected back out of the eye 20. The reflected light passes back through the beam splitter 40, through an objective lens 50, and is focused by an eyepiece lens 60 through the pupil 70 of the smartphone camera onto the image plane 80 of the smartphone camera.

In some embodiments, the pupil 30 of the eye 20 has a diameter of about 4 mm, the objective lens 50 has an effective focal length of about 8 mm and is positioned about 16 mm from the pupil 30. The eyepiece lens 60 may be positioned about 10 mm in front of an intermediate image plane 90. The camera pupil may have a diameter of about 2 mm and may be positioned about 38 mm from the pupil 30.

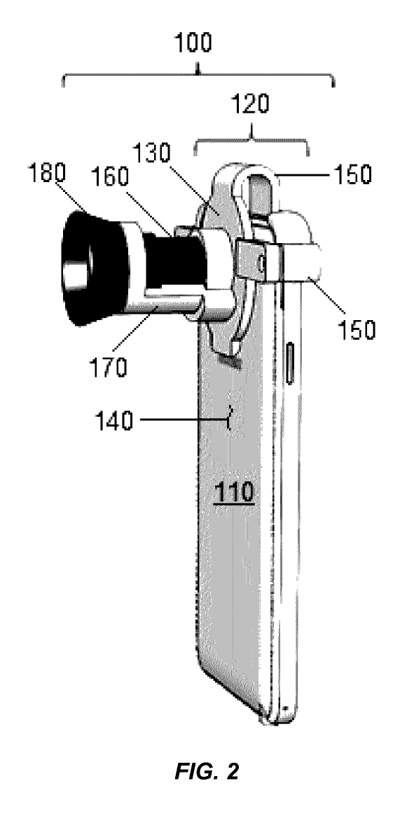

An embodiment of a fixture housing the optics of the disclosed fundoscopy system is a clamp-on smartphone attachment that secures the magnifying optics on the camera line-of-sight, offering a degree of adjustment for focus and illumination, and a flexible eyepiece to align the subject. An embodiment of the fixture 100 is illustrated mounted on a smartphone 110 in FIG. 2. The fixture 100 includes a mount 120 having a flat base 130 configured to be disposed against the rear side 140 or rear surface of the smartphone 110. The fixture 100 further includes a plurality of legs 150, for example, three legs as illustrated in FIG. 2, that fit around the body of the smartphone 110 and hold the base 130 in place against the rear side 140 of the smartphone 110. The legs 150 may be formed from a resilient material that deforms slightly when the fixture 100 is mounted on the smartphone 110 and exerts pressure on the front and rear surfaces of the smartphone 110 to hold the fixture 100 in place on the smartphone 110. A barrel 160 including the beam splitter 40, objective lens 50, and eyepiece lens 60 is secured in a barrel mount 170 coupled to the front of the flat base 130. The barrel mount 170 holds the optics (beam splitter 40, objective lens 50, and eyepiece lens 60) in place aligned in front of the pupil 70 of the smartphone camera. An eyepiece 180, which in some embodiments is made of a flexible material, for example, rubber is mounted to the front of the barrel mount 170. In some embodiments, one or more of the beam splitter 40, objective lens 50, and eyepiece lens 60 include an anti-reflective coating to help reduce reflections that may degrade the quality of an image of the retina taken by the smartphone camera through the optics mounted in the fixture. In some embodiments, a manual adjustment actuator may be provided to adjust the position of the barrel 160 within the barrel mount 170 and/or to adjust a position of one or more of the optics elements relative to one another to provide for adjustments to focus of the system. In some embodiments, the smartphone 110 may be programmed with customized software to adjust the manner in which the camera operates to focus when using the fixture 100 to obtain an image of a subject's retina, for example, to fix the camera lens-to-CCD distance for imaging objects at infinity.

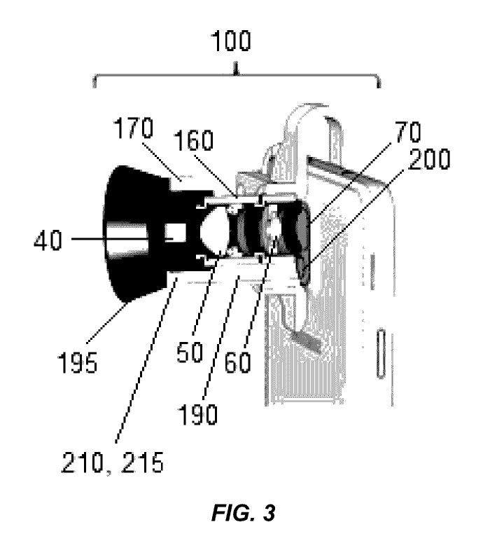

FIG. 3 illustrates a partial cross-sectional view of the fixture 100 and associated optics. As can be seen, the fixture 100 aligns the beam splitter 40, objective lens 50, and eyepiece lens 60 directly in front of the pupil 70 of the smartphone camera. The barrel mount 170 further includes a light guide 190, which may be an open conduit or may include a fiber optic light pipe, aligned with the illumination source, for example, LED 200 of the smartphone 110 and having an axis normal to the rear face 140 of the smartphone and parallel to a path of light through the optics. The light from the LED 200 of the smartphone 110 passes through the light guide 190, is reflected by a mirror 210 disposed at a distal end of the light guide 190, though light guide 195, which may be an open conduit or may include a fiber optic light pipe, and into the beam splitter 40, and is directed by the beam splitter 40 out of the front of the barrel 160 toward the eye 20 of a subject. Light guide 190 is substantially or fully perpendicular to light guide 195. Mirror 210 is angled at about or exactly 45 degrees relative to the axes of light guides 190 and 195. It should be appreciated that in some embodiments mirror 210 is not necessary. For example, surface 215 upon which mirror 210 is disposed in some embodiments, may be sufficiently reflective, for example, formed from a bright white polymeric material or a metallic material, such that an acceptable amount of light will be reflected from the LED 200 of the smartphone 110 to the beam splitter 40 out of the front of the barrel 160 toward the eye 20 of a subject.

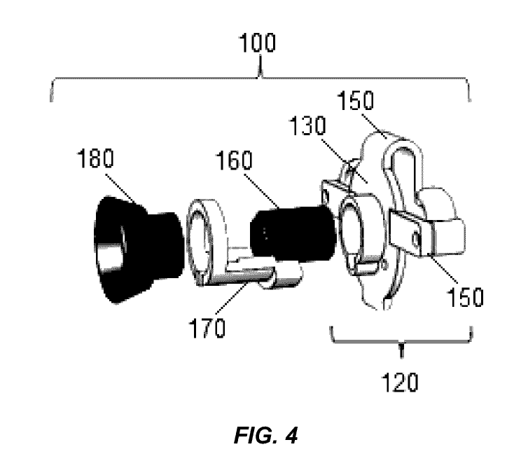

FIG. 4 illustrates the fixture 100 in an exploded view separated from the smartphone 110. The fixture 100 may be formed from, for example, polypropylene, polyethylene, polyvinyl chloride, or any other suitable polymer or metallic material. Components of the fixture 100, for example, the base 130, legs 150, and/or barrel mount 170 may be formed by injection molding, 3D printing, or any other suitable process. Components of the fixture 100, for example, the base 130, legs 150, and/or barrel mount 170 may be formed as separate elements and later joined by, for example, an adhesive and/or snaps or other fasteners known in the art, or may be formed as an integral one piece structure.

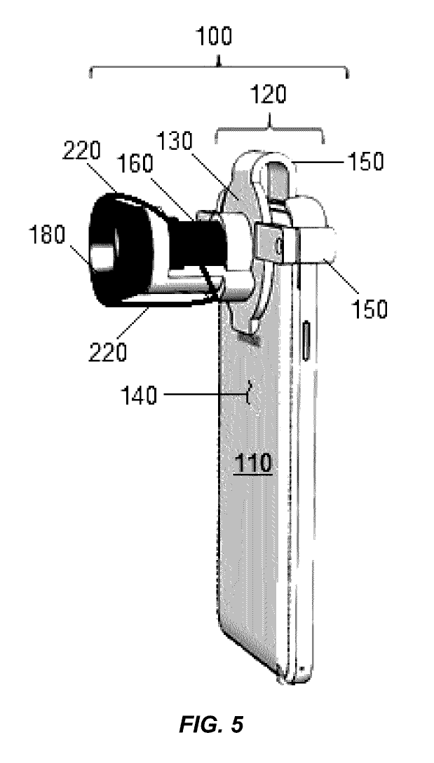

In other embodiments, alternative or additional illumination systems may be utilized. For example, as illustrated in FIG. 5, one or more light guides 220, for example, short lengths of fiber optic cable, may be utilized to direct light from the LED 200 of the smartphone 110 to positions at the periphery of the eyepiece 180 to illuminate the retina 10 at an angle, rather than in a direction normal to the retina 10. The arrangement illustrated in FIG. 5 may help reduce the amount of light reflected from the surface of the eye 10 into the pupil 70 of the smartphone camera, thus improving image quality.

It should be appreciated that different smartphones have different arrangements of cameras and illumination sources. Different embodiments of the fixture 100 may be modified based on a type of smartphone that they are intended to be used with such that the fixture 100 will position the optics and light guide(s) in appropriate locations in front of the camera pupil and illumination source of the respective type of smartphone. It should also be appreciated that in alternative embodiments, the fixture 100 may be mounted on the front, or screen side, of a smartphone having a front facing camera and illumination source. In such embodiments an individual may use the screen of the smartphone to display an image of the retina prior to taking a photograph of the retina to allow the individual to properly position the smartphone and take a photograph of his/her own retina.

In some embodiments, a method of obtaining an image of a subject's retina may comprise obtaining a smartphone having a camera and illumination source and a fixture 100 as described above that is configured for use with the particular smartphone. The subject or another individual, for example, an assistant, friend, family member, or healthcare professional, for example, a doctor or a nurse may utilize the smartphone and fixture to capture an image of one or both of the subject's retinas. The method involves mounting the fixture on the smartphone such that the optics are aligned with the pupil of the camera of the smartphone and the light guide 190 is aligned with the illumination source (e.g., LED) of the smartphone. The subject or other individual positions the smart phone and fixture in front of the pupil of one of the eyes of the subject, allows the smartphone camera to focus on the retina of the eye of the subject, and then actuates the smartphone camera to capture an image of the retina. The image may be examined on the screen of the smartphone for signs of diabetic retinopathy or may be transferred to another diagnostic system or sent by e-mail or another form of electronic communication to a doctor or diagnostic system at a location remote from the subject for analysis.

### Example

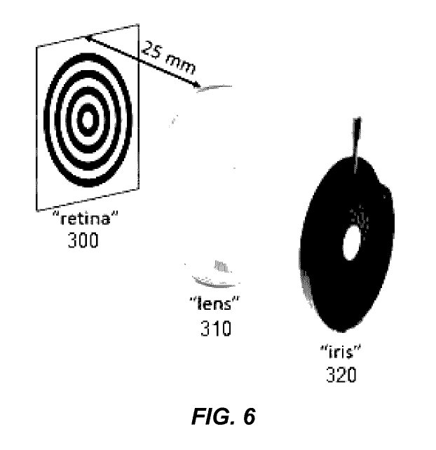

Preliminary tests to validate the optical system design were performed on an optical test bench using off-the-shelf components to simulate a human eye. The retina, lens, and iris of a human eye were mimicked on a standard optical table by placing a printed target pattern 300 (the “retina”) at the focal point of a plano-convex lens 310 (focal length=25 mm) simulating the lens of a human eye and mounting a mechanical iris 320 on the opposite side of the lens 310. (See FIG. 6.) Imaging and illumination optics as described above (beamsplitter, objective lens, and eyepiece lens) were installed between the simulated eye and a smartphone camera using optomechanical mounts.

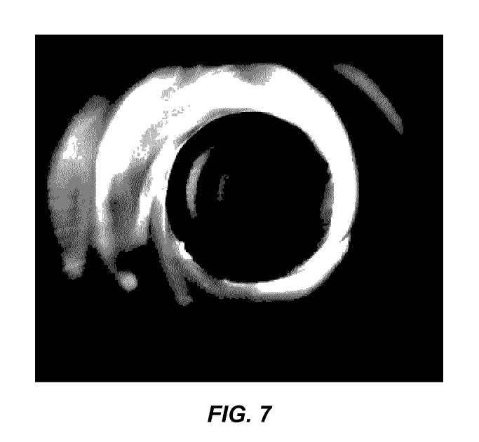

Clear images of the “retina,” shown in FIG. 7, were straightforwardly acquired with the smartphone using the image relay system. It was necessary to configure the smartphone camera software to fix the lens-to-CCD distance for imaging objects at infinity, otherwise the auto-focus software feature would adjust the lens-to-CCD distance to image an intermediate object (e.g., an optomechanical mount for one of the lenses in the system). This example shows that the disclosed imaging system is compatible with smartphone technology to provide images of human retinas.

Having now described some illustrative embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention. In particular, although many of the examples presented herein involve specific combinations of method acts or system elements, it should be understood that those acts and those elements may be combined in other ways to accomplish the same objectives.

Those skilled in the art should appreciate that the parameters and configurations described herein are exemplary and that actual parameters and/or configurations will depend on the specific application in which the systems and techniques of the invention are used. Those skilled in the art should also recognize or be able to ascertain, using no more than routine experimentation, equivalents to the specific embodiments of the invention. It is therefore to be understood that the embodiments described herein are presented by way of example only and that, within the scope of the appended claims and equivalents thereto; the invention may be practiced otherwise than as specifically described.

Moreover, it should also be appreciated that the invention is directed to each feature, system, subsystem, or technique described herein and any combination of two or more features, systems, subsystems, or techniques described herein and any combination of two or more features, systems, subsystems, and/or methods, if such features, systems, subsystems, and techniques are not mutually inconsistent, is considered to be within the scope of the invention as embodied in the claims. Further, acts, elements, and features discussed only in connection with one embodiment are not intended to be excluded from a similar role in other embodiments.

As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A fundoscopy system comprising a fixture supporting optics configured to be mounted to a surface of a smartphone and align the optics with a pupil of a camera of the smartphone and to align a light guide with an illumination source of the smartphone, the light guide configured to direct light from the illumination source into an eye of a subject, the fixture further configured to direct light reflected from a retina of the eye through the optics and into the pupil of the camera of the smartphone.

2. The system of claim 1, wherein the fixture includes a mount having a flat base configured to be disposed against a rear surface of the smartphone.

3. The system of claim 2, wherein the fixture further comprises a plurality of legs that fit around a body of the smartphone and hold the base in place against the rear surface of the smartphone.

4. The system of claim 3, wherein the plurality of legs are formed from a resilient material that deforms when the fixture is mounted on the smartphone and exerts pressure on front and rear surfaces of the smartphone to hold the fixture in place on the smartphone.

5. The system of claim 1, wherein the optics include a beam splitter, objective lens, and eyepiece lens.

6. The system of claim 5, wherein the optics are disposed in a barrel and the fixture further includes a barrel mount configured to retain the barrel.

7. The system of claim 5, wherein one or more of the beam splitter, objective lens, and eyepiece lens has an anti-reflective coating.

8. The system of claim 1, further comprising an eyepiece disposed on a front of the fixture.

9. The system of claim 1, wherein the light guide is defined in a body of the fixture and has an axis parallel to a path of light through the optics.

10. The system of claim 1, wherein the light guide terminates at a reflector disposed at an angle of approximately 45 degrees relative to the axis of the light guide.

11. The system of claim 10, wherein the reflector is a mirror.

12. The system of claim 10, wherein the reflector is a reflective inner surface of the fixture.

13. The system of claim 10, wherein the fixture further includes a second light guide extending from the reflector to a beam splitter included in the optics.

14. The system of claim 13, wherein the second light guide is substantially perpendicular to the light guide.

15. The system of claim 1, wherein the fixture further includes one or more light guides configured to direct light from the illumination source of the smartphone to one or more positions at a periphery of an eyepiece of the fixture.

16. The system of claim 15, wherein the one or more light guides comprise fiber optic cables.

17. A method of imaging a retina of a subject, the method comprising:
mounting a fixture supporting optics and a light guide to a surface of a smartphone;
aligning the optics with a pupil of a camera of the smartphone;
aligning the light guide with an illumination source of the smartphone, the light guide directing light from the illumination source into an eye of the subject disposed in front of the fixture; and
receiving light reflected from the retina of the eye through the optics and into the pupil of the camera of the smartphone.

18. The method of claim 17, further comprising capturing an image of the retina with the camera of the smartphone.

19. The method of claim 18, further comprising analyzing the image for signs of diabetic retinopathy.

20. The method of claim 18, further comprising electronically transmitting the image to a remote location for analysis.
